(19) United States
(12) Patent Application Publication
(10) Pub. No.: US 2025/0387137 A1
(43) Pub. Date: Dec. 25, 2025

(54) INSERTION DEVICE

(71) Applicant: Roche Diabetes Care, Inc., Indianapolis, IN (US)

(72) Inventor: Kirill Sliozberg, Mannheim (DE)

(73) Assignee: Roche Diabetes Care, Inc., Indianapolis, IN (US)

(21) Appl. No.: 19/258,107

(22) Filed: Jul. 2, 2025

Related U.S. Application Data
(63) Continuation of application No. PCT/EP2024/050756, filed on Jan. 15, 2024.

(30) Foreign Application Priority Data
Jan. 16, 2023 (EP) EP23151725.1

(51) Int. Cl. A61B 17/34; A61B 5/145

(52) U.S. Cl. CPC A61B 17/3468; A61B 5/14503; A61B 2560/063

(57) ABSTRACT

An insertion device for at least partially inserting a medical device into a body tissue of a subject is disclosed. The insertion system comprises: at least one medical device holding structure; an insertion component movable with the medical device holding structure from a distal position to a proximal position; at least one locking member having a predefined tensile strength Fₜᵤ, wherein the locking member is configured for holding the medical device holding structure and the insertion component in the distal position; and at least one pusher configured for applying an external force to overcome the predefined tensile strength of the locking member.

### CROSS-REFERENCE TO RELATED APPLICATIONS

The present application is a U.S. Continuation of International Application No. PCT/EP2024/050756, filed on Jan. 15, 2024, which claims the benefit of and the priority to European Patent Application No. EP 23151725.1, filed on Jan. 16, 2023, the entire contents of which are incorporated herein by reference in their entirety for all purposes.

### TECHNICAL FIELD

The invention relates to an insertion device, an insertion system and a method for at least partially inserting a medical device into a body tissue of a subject. The medical device may specifically be configured for detecting at least one analyte in a body fluid of the subject. The insertion device, the insertion system and the method may be applied in the field of continuous monitoring of the analyte in the body fluid of the subject, specifically in the field of home care and in the field of professional care, such as in hospitals. Other applications, however, are also feasible.

### BACKGROUND ART

Monitoring certain body functions, more particularly monitoring one or more analyte concentrations such as one or more metabolite concentrations in a body fluid of a subject plays an important role in the prevention and treatment of various diseases. Such analytes can include by way of example, but not exclusively, glucose, lactate, cholesterol or other types of analytes and metabolites. Without restricting further possible applications, the invention will be described in the following text with reference to glucose monitoring. However, additionally or alternatively, the invention can also be applied to other types of analytes.

Subcutaneous analyte sensing device demand a corresponding insertion device. Usually, a subcutaneous analyte sensor is based on a plastic substrate and, thus, cannot be inserted by itself and demands a metallic cannula. The insertion cannula typically possesses a special void for placing the sensor in it. During the insertion process, the cannula containing the sensor, penetrates the skin and retracts leaving the sensor inserted. One of the important roles of the insertion device may be moving the cannula with a well-defined initial speed, to successfully penetrate the skin without causing excessive pain. In order to achieve an initial momentum, the insertion mechanism usually possesses a mechanical assembly, which releases the cannula after some predefined force threshold is exceeded during the insertion process.

For example, a user places the insertion mechanism, with the insertion cannula and sensor in it, on the skin surface and presses on top of the whole assembly. Once the applied force is sufficient, the mechanism releases the cannula and the insertion takes place. It is important that the force to be overcome is well defined. Otherwise, if the cannula momentum is too low, the insertion would either not work properly, since the cannula will not penetrate the skin completely, or would cause excessive pain, due to the slow penetration. If the threshold force is set too high, the excessive pressure on the insertion device would compress the tissue under it and cause the cannula to penetrate the skin much deeper, as needed, which, again, would cause an excessive pain and the wound will be unneeded deep, which may negatively influence the sensor performance due to physiological reaction.

The typical mechanism defining the release force may be based on springe plastic bars, which press against another part. Once sufficient force is applied, the bars may be pressed out from their positions suddenly and thus the cannula gets its momentum. In order to grant a reproducible and well-controlled release force, the shape and measures of these parts have to be highly precise, which by an issue for mass manufacturing. Moreover, the flexibility of these plastic parts may strongly depend on the current temperature as well as on the age of these parts, as mechanical properties of plastics, foremost such as flexibility, may change drastically over time.

EP 3 474 729 A describes a medical device for transcutaneously inserting an insertable element into a body tissue. The medical device comprises: —at least one insertable element, wherein the insertable element comprises at least one in vivo distal end for subcutaneous insertion and at least one ex vivo proximal end; —at least one insertion cannula for subcutaneously inserting the insertable element, the insertion cannula having a lumen which fully or partially is enclosed by a wall of the insertion cannula, wherein the insertable element is received in the lumen, wherein the wall comprises at least one shape memory alloy, wherein the insertion cannula is stored in a first shape configuration, wherein the insertion cannula is configured to be transformable into a second shape configuration for insertion.

US 2018/0353110 A1 describes devices, systems, methods and kits for releasably mounting a medical device on the body or skin of a user. Embodiments include a holder or mounting unit or structure that retains a medical device in a fixed position on a body part of a user or host, such as on the surface of the skin, and/or provides physical and/or electrical coupling to one or more additional components which may be operatively positioned above and/or below the surface of the skin.

U.S. Pat. No. 10,827,955 B2 describes a sensor system including a base having a distal side configured to face towards a person's skin. An adhesive can couple the base to the skin. A transcutaneous analyte measurement sensor can be coupled to the base and can be located at least partially in the host. A transmitter can be coupled to the base and can transmit analyte measurement data to a remote device.

### Problem to be Solved

It is therefore desirable to provide an insertion device, an insertion system and a method for at least partially inserting a medical device into a body tissue of a subject which at least partially address the above-mentioned technical challenges. Specifically, an insertion device, an insertion system and a method for at least partially inserting a medical device into a body tissue of a subject are desirable which allow a safe and user-friendly handling of the insertion device while ensuring a reliable insertion of a medical device into a body tissue of a subject.

### SUMMARY

This problem is addressed by an insertion device, an insertion system and a method for at least partially inserting a medical device into a body tissue of a subject with the features of the independent claims. Advantageous embodiments which might be realized in an isolated fashion or in any arbitrary combinations are listed in the dependent claims as well as throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present invention, an insertion device for at least partially inserting a medical device into a body tissue of a subject is disclosed. The insertion device comprises at least one medical device holding structure. Further, the insertion device comprises an insertion component movable with the medical device holding structure from a distal position to a proximal position. Further, the insertion device comprises at least one locking member having a predefined tensile strength Ft. The locking member is configured for holding the medical device holding structure and the insertion component in the distal position. Further, the insertion device comprises at least one pusher configured for applying an external force to overcome the predefined tensile strength of the locking member.

The term “medical device” as used herein is a broad term and is to be given its ordinary and customary meaning to a person of ordinary skill in the art and is not to be limited to a special or customized meaning. The term specifically may refer, without limitation, to an arbitrary element or article being configured for use in the field of medical technology, exemplarily in the field of medical analytics or medical diagnostics. The medical device may be configured for performing at least one medical function and/or for being used in at least one medical process, such as one or more of a therapeutic process, a diagnostic process or another medical process.

The medical device may be configured to be mounted on a skin site of an extremity of the subject. The extremity may be selected from the group consisting of: an arm, exemplarily an upper arm; a stomach; a shoulder; a back; hip; a leg. Exemplarily, the extremity may be the upper arm. However, also other applications may be feasible.

The medical device may comprise at least one component which may be configured to stay outside of the body tissue. Further, the medical device may comprise at least one insertable portion. The insertable portion may be configured for being inserted into the body tissue of the subject. Exemplarily, the insertion device may be configured for inserting the insertable portion of the medical device into the body tissue of the subject. For example, the insertion device for at least partially inserting a medical device into a body tissue of a subject may be configured for inserting the insertable portion of the medical device into the body tissue of the subject.

As outlined above, the medical device may comprise the at least one insertable portion. The medical device, exemplarily the insertable portion of the medical device, may comprise at least one device selected from the group consisting of: an analyte sensor for detecting at least one analyte in a body fluid of the subject, an infusion cannula and stimulating electrodes. Other embodiments may be feasible.

The analyte sensor for detecting at least one analyte in a body fluid of a subject, such as in a body fluid contained in a body tissue of the subject, may be configured for being used in qualitatively and/or quantitatively detecting the at least one analyte.

The term “analyte” as used herein is a broad term and is to be given its ordinary and customary meaning to a person of ordinary skill in the art and is not to be limited to a special or customized meaning. The term specifically may refer, without limitation, to a chemical and/or biological substance which takes part in the metabolism of the body of the subject. Exemplarily, the analyte may be a metabolite or a combination of two or more metabolites. As an example, the analyte may be selected from the group consisting of: glucose, lactate, triglycerides, cholesterol. Still, other analytes or combinations of two or more analytes may be detected. The body tissue exemplarily may be or may comprise fatty tissue and/or interstitium. Other types of body tissue, however, are feasible.

The term “analyte sensor” as used herein is a broad term and is to be given its ordinary and customary meaning to a person of ordinary skill in the art and is not to be limited to a special or customized meaning. The term specifically may refer, without limitation, to a sensor which is capable of qualitatively or quantitatively detecting the presence and/or the concentration of the at least one analyte. The analyte sensor may be an electrochemical analyte sensor. The analyte sensor may comprise at least two electrodes. Exemplarily, the analyte sensor may comprise at least one two-electrode sensor. The two-electrode sensor may comprise precisely two electrodes, such as a working electrode and at least one further electrode such as a counter electrode, e.g. a working electrode and a combined counter/reference electrode. The working electrode may comprise a working electrode pad and, optionally, at least one test chemical disposed thereon. The counter electrode may comprise a counter electrode pad. Additionally and optionally, one or more redox materials may be disposed thereon. The analyte sensor may further comprise one or more leads for electrically contacting the electrodes. The leads may, during insertion or at a later point in time, be connected to one or more electronic components. For example, the leads may already be connected to the electronic components before insertion of the analyte sensor.

For example, the analyte sensor may be a needle-shaped or a strip-shaped analyte sensor having a flexible substrate and the electrodes disposed thereon. As an example, the analyte sensor may have a total length of 5 mm to 50 mm, e.g. a total length of 7 mm to 30 mm. The term “total length” within the context of the present invention relates to the overall length of the analyte sensor which means a portion of the analyte sensor which is inserted and the portion of the analyte sensor which may stay outside of the body tissue. The portion of the analyte sensor which is inserted is also called the in-vivo portion, the portion of the analyte sensor which may stay outside of the body tissue is also called the ex vivo portion. For example, the in vivo portion has a length in the range from 3 mm to 12 mm. The analyte sensor may further comprise a biocompatible cover, such as a biocompatible membrane which fully or partially covers the analyte sensor and which prevents the test chemical from migrating into the body tissue and which allows for a diffusion of the body fluid and/or the analyte to the electrodes. Other embodiments of electrochemical analyte sensors, such as three-electrode sensors, may be feasible. For example, the three-electrode sensor may comprise, in addition to the working electrode and the counter electrode, a reference electrode.

In another embodiment, the analyte sensor may be an optical analyte sensor. For example, the analyte sensor may comprise a flexible light guide with glucose sensitive coating at its end and/or a tube like carrier with functional elements at inner or outer walls. Other embodiments of the analyte sensor may be possible, too.

The term “infusion cannula” as used herein is a broad term and is to be given its ordinary and customary meaning to a person of ordinary skill in the art and is not to be limited to a special or customized meaning. The term exemplarily may refer, without limitation, to a hollow tube configured for delivering and/or infusing a medication into the body tissue of the subject, e.g. for delivering and/or infusing insulin into the body tissue of the subject.

As outlined above, the medical device may comprise the at least one component which may be configured to stay outside of the body tissue. Exemplarily, the medical device may comprise at least one housing. One or more electronic components may be received in the housing. The term “housing” as used herein is a broad term and is to be given its ordinary and customary meaning to a person of ordinary skill in the art and is not to be limited to a special or customized meaning. The term exemplarily may refer, without limitation, to an element or component having at least one interior space and at least one wall fully or partially surrounding the at least one interior space and providing protection to the interior space, such as one or more of a mechanical protection and a protection against environmental influences such as one or more of moisture, oxygen and microbial contaminations. The housing may generally be adapted to fully or partially surround and/or receive one or more elements in order to provide one or more of a mechanical protection, a mechanical stability, an environmental protection against moisture and/or ambient atmosphere, a shielding against electromagnetic influences or the like. The housing may also provide a basis for attachment and/or holding one or more further components or elements.

Exemplarily, the housing may be configured for holding one or more electronic components. Thus, the housing may also be referred to as an electronics unit. Exemplarily, in case the medical device is a medical device for detecting the analyte in the body fluid, the electronics unit may be configured for one or more of determining and/or controlling a detection of the analyte and/or transmitting measurement data to another component. Exemplarily, the electronics component may be configured for one or more of performing a measurement with the sensor, performing a voltage measurement, performing a current measurement, recording sensor signals, storing measurement signals and/or measurement data, transmitting sensor signals to another component. Thus, the electronics unit exemplarily may comprise at least one of: a voltmeter, an ammeter, a potentiostat, a voltage source, a current source, a signal receiver, a signal transmitter, an analog-digital converter, an electronic filter, a data storage device, an energy storage device. The housing may exemplarily be embodied as a closed electronics unit. The analyte sensor may be partially enclosed by the housing.

In an embodiment, the housing may comprise at least one patch. The patch exemplarily may comprise a plate which may be used as a support for other components of the medical device such as of the electronic components. Further, the patch may be configured for attaching the components of the medical device to the skin site of the subject. For this purpose, the patch may comprise at least one adhesive surface and/or at least one adhesive strip or plaster.

However, the housing may not comprise a patch. In this case, the housing may further be configured for direct attachment to the skin site of the subject. The housing may comprise at least one attachment component which is capable of forming a connection to the skin site, such as at least one adhesive surface and/or at least one adhesive strip or plaster.

The term “subject” as used herein is a broad term and is to be given its ordinary and customary meaning to a person of ordinary skill in the art and is not to be limited to a special or customized meaning. The term exemplarily relates to a person intending to monitor an analyte value, such as a glucose value, in the person's body tissue and/or to deliver medication, such as insulin, into the person's body tissue. In an embodiment, the term specifically may refer, without limitation, to a person using the insertion device. However, in an embodiment, the person using the insertion device is different from the subject. For example, the medical device may be inserted by a person different from the subject into the subject's body tissue. For example, the subject may be a patient suffering from a disease, such as diabetes. The subject may also be referred to as user or as patient.

The term “inserting” as used herein is a broad term and is to be given its ordinary and customary meaning to a person of ordinary skill in the art and is not to be limited to a special or customized meaning. The term specifically may refer, without limitation, to an action or process of one or more of transcutaneously or subcutaneously implanting and/or positioning the medical device, exemplarily the insertable portion of the medical device, into the body tissue of the subject. The medical device may fully or partially be inserted into the body tissue. The insertion of the medical device may be performed by using the insertion device. After insertion, the medical device or at least a part of the medical device may remain in the body tissue of the subject for a predetermined period of time, such as for several hours, exemplarily for one or more days, such as for up to one week, or such as for up to two weeks or even more. The medical device may exemplarily be configured for continuously monitoring and/or detecting the analyte in the body fluid of the subject.

The term “distal position” as used herein is a broad term and is to be given its ordinary and customary meaning to a person of ordinary skill in the art and is not to be limited to a special or customized meaning. The term specifically may refer, without limitation, to a position specification indicating a position of the insertion device and/or any parts thereof and/or of the medical device in relation to the subject in which the insertion component, the medical device holding structure, the medical device and/or the pusher are furthermost from the proximal side of the insertion device. Exemplarily, for inserting the medical device, the insertion device may be brought into contact with a skin site of the subject. The distal position may refer to a position being distanced to the skin site of the subject. The distal position may be an initial position prior to the insertion movement of the insertion device and/or any parts thereof. Each component of the insertion device may have its own and/or individual distal position. For example, the pusher, the medical device holding structure, the insertion component and/or the medical device may have their own and/or individual distal positions, respectively. Prior to insertion, the pusher, the medical device holding structure, the insertion component and/or the medical device may be in their distal position and may be ready for inserting the medical device into the body tissue of the subject.

The term “proximal position” as used herein is a broad term and is to be given its ordinary and customary meaning to a person of ordinary skill in the art and is not to be limited to a special or customized meaning. The term specifically may refer, without limitation, to a position specification indicating a position of the insertion device and/or any parts thereof and/or of the medical device in relation to the subject in which the insertion component, the medical device holding structure, the medical device and/or the pusher closest to the proximal side of the insertion device. Exemplarily, for inserting the medical device, the insertion device may be brought into contact with the skin site of the subject. The proximal position may refer to a position being in close proximity to the skin site of the subject. Each component of the insertion device may have its own and/or individual proximal position. For example, the insertion component, the medical device holding structure, the medical device and/or the pusher may have their own and/or individual proximal positions, respectively. In case the insertion component, the medical device holding structure, the medical device and/or the pusher are in their proximal position, the medical device may be inserted into the body tissue of the subject. During insertion of the medical device, the medical device may be in contact with the skin site of the subject and, thus, may be in its proximal position.

The term “insertion device” as used herein is a broad term and is to be given its ordinary and customary meaning to a person of ordinary skill in the art and is not to be limited to a special or customized meaning. The term specifically may refer, without limitation, to a device configured for inserting the medical device into the body tissue. The insertion device may be configured for transcutaneously or subcutaneously inserting the medical device into the body tissue, such as by performing an incision or a puncture in a skin of the subject and by transferring the medical device fully or partially into the body tissue. The insertion device may be removed fully or partially after inserting the medical device at least partially into the body tissue of the subject.

As outlined above, the insertion device comprises the at least one insertion component. The term “insertion component” as used herein is a broad term and is to be given its ordinary and customary meaning to a person of ordinary skill in the art and is not to be limited to a special or customized meaning. The term specifically may refer, without limitation, to an arbitrary element which may be insertable at least partially into the body tissue, particularly in order to deliver or to transfer a further element. The insertion component may be configured for supporting the insertion of the medical device or the insertion of a part of the medical device. Exemplarily, the insertion component may be configured for supporting the insertion of the insertable portion of the medical device. The insertion component may comprise a tip or a sharp end for inserting the medical device, exemplarily the insertable portion of the medical device, into the body tissue. The insertion component may be or may comprise an insertion cannula or an insertion needle. In the context of the present invention, the insertion component is considered as part of the insertion device. However, in particular during manufacturing of an insertion system the insertion component may also be considered as a part of the medical device.

The term “insertion cannula” as used herein is a broad term and is to be given its ordinary and customary meaning to a person of ordinary skill in the art and is not to be limited to a special or customized meaning. The term specifically may refer, without limitation, to a hollow needle which may be at least partially slotted. The medical device may be received within the insertion cannula, such as within a lumen of the insertion cannula. The term “insertion needle” as used herein is a broad term and is to be given its ordinary and customary meaning to a person of ordinary skill in the art and is not to be limited to a special or customized meaning. The term specifically may refer, without limitation, to a compact needle, exemplarily without a slot and without any hollow parts. The medical device may be received on an outer surface of the insertion needle.

After insertion, the medical device, exemplarily the insertable portion of the medical device, may remain at least partially in the body tissue of the subject. The insertion component, however, may be retracted from the body tissue of the subject into the insertion device after inserting the medical device.

The insertion device may be configured for retracting the insertion component from the body tissue of the subject into the insertion device housing. The retraction may be performed by using at least one retraction mechanism. The retraction mechanism may be designed to be coupled with the insertion force defining principle The insertion device may comprise at least one retractor. For example, the locking member, such as the fiber, may be spanned between the not moving insertion device part and the retractor. During the insertion the fiber brakes, the insertion component gets inserted, and the retractor can subsequently retract the insertion component. Such a configuration may allow using one single fiber in the middle. The insertion device may further comprise at least one safety mechanism for preventing unintentional inserting the insertion component and/or reuse of the insertion component.

Further, as outlined above, the insertion device comprises the at least one medical device holding structure. The term “medical device holding structure” as used herein is a broad term and is to be given its ordinary and customary meaning to a person of ordinary skill in the art and is not to be limited to a special or customized meaning. The term specifically may refer, without limitation, to an arbitrary element configured for at least partially enclosing a medical device and/or for at least partially holding the medical device in a desired position. Exemplarily, the medical device holding structure may comprise at least one receptacle and the medical device may be at least partially received in the receptacle. For example, medical device holding structure may comprise a rotational symmetric hollow center. The rotational symmetric hollow center may exemplarily be open to a proximal end of the medical device holding structure. The medical device may be arrangable at the proximal end of the medical device holding structure. The medical device holding structure may also be referred to as insertion sleeve.

Further, the medical device holding structure may comprise one or more holding elements. As described above, the medical device may comprise the housing. The housing may be held by the holding elements. The term “holding element” as used herein is a broad term and is to be given its ordinary and customary meaning to a person of ordinary skill in the art and is not to be limited to a special or customized meaning. The term specifically may refer, without limitation, to an element of the medical device holding structure which is configured for engaging with the housing of the medical device, exemplarily of a surface of the medical device, for the purpose of supporting the housing and keep the housing in position. The holding element may be or may comprise at least one finger, gripper, hook, pincer or the like. Also other embodiments may be feasible. The holding element may be arranged at an inner structure of the medical device holding structure, exemplarily at an inner structure of the receptacle of the medical device holding structure.

The insertion device may be separable from the medical device. Exemplarily, the medical device holding structure may be separable from the medical device. The term “separable” as used herein is a broad term and is to be given its ordinary and customary meaning to a person of ordinary skill in the art and is not to be limited to a special or customized meaning. The term specifically may refer, without limitation, to a property of the insertion device of being disengageable from the medical device. The disengagement of the insertion device from the medical device may be supported by the subject. Exemplarily, the subject may exert a force onto the insertion device such as by pulling on the insertion device, exemplarily on the pusher. The medical device may exemplarily comprise the plaster and may be attached to the skin site of the subject via adhesive forces. The separation of the insertion device from the medical device may be typically carried out after the insertion of the medical device. As outlined above, the medical device holding structure may comprise the holding elements. The housing of the medical device may be held by the holding elements. The separation of the insertion device from the medical device may include a releasing of the medical device, exemplarily of the housing of the medical device, by the holding elements.

As outlined above, the insertion device comprises the at least one pusher. The term “pusher” as used herein is a broad term and is to be given its ordinary and customary meaning to a person of ordinary skill in the art and is not to be limited to a special or customized meaning.

The term specifically may refer, without limitation, to an arbitrary shaped element which is configured for moving an object in a specified direction by exerting force. Exemplarily, the element may be pressed with force in order to move the object in the specified direction. Further, the pusher may be configured for fully or partially enclosing one or more components of the insertion device and/or for providing protection for these one or more components, such as against mechanical influence and/or humidity. Thus, the pusher may also be referred to as cap. Further, the pusher may be or may comprise at least one cap.

The pusher may surround and/or may enclose fully or partially one or more further components, such as the medical device holding structure, the medical device and the locking member. The term “at least partially surround”, also referred to as “at least partially enclose”, as used herein is a broad term and is to be given its ordinary and customary meaning to a person of ordinary skill in the art and is not to be limited to a special or customized meaning. The term specifically may refer, without limitation, to embodiments wherein the pusher fully surrounds the one or more further components of the insertion device, and to embodiments wherein the pusher may surround at least a part of the one or more further components. Exemplarily, the medical device holding structure, the medical device and/or the locking member may be at least partially received in the pusher.

The insertion device may comprise at least one insertion device housing. The term “insertion device housing” as used herein is a broad term and is to be given its ordinary and customary meaning to a person of ordinary skill in the art and is not to be limited to a special or customized meaning. The term specifically may refer, without limitation, to an enclosure of one or more components configured for guiding at least one movement of components enclosed by the insertion device housing, exemplarily of the medical device holding structure and the medical device. The insertion device housing may be partially enclosed by the pusher. The insertion device housing may also be referred to as guide sleeve.

The insertion device housing may be essentially rotationally symmetric, exemplarily in accordance with the symmetry of the pusher of the insertion device, e.g. of the inner structure of the pusher of the insertion device. For example, in case the pusher may have an axial rotational symmetry about an axis such as a cylinder axis or axis of extension, the insertion device housing may have a similar axial rotational symmetry. Exemplarily, the insertion device housing may have a shape which is adapted to a shape of the medical device.

The insertion device housing may be stationary with respect to the pusher of the insertion device. For example, when using the insertion device, the pusher may be configured for sliding into the insertion device housing. The insertion device housing, e.g. a proximal end of the insertion device housing, may be in contact with the subject's skin when the insertion device is used. Thus, the insertion device housing may also be referred to as frame or as sheath.

As outlined above, the insertion device comprises the at least one locking member. The term “locking member” as used herein is a broad term and is to be given its ordinary and customary meaning to a person of ordinary skill in the art and is not to be limited to a special or customized meaning. The term specifically may refer, without limitation, to an arbitrary device which is configured for controlling a movement of another object. Exemplarily, the locking member may be configured for holding the other object in a desired position. Further, the locking member may be configured for releasing the other object under controlled conditions. For the purpose of releasing the other object, the locking member may exemplarily be configured for breaking or rupturing. Thus, the locking member may also be referred to as breaking element or breaking component. The locking member may exemplarily be or may comprise at least one elongate element. The locking member may exemplarily be or may comprise at least one fiber. Further details on the fiber are given below in more detail.

As outlined above, the locking member has a predefined tensile strength. The term “tensile strength” as used herein is a broad term and is to be given its ordinary and customary meaning to a person of ordinary skill in the art and is not to be limited to a special or customized meaning. The term specifically may refer, without limitation, to one of several strength parameters of a material. The term specifically refers to a maximum mechanical tensile stress that the material can withstand while being stretched or pulled before breaking. The dimension of the tensile strength is force per area with the measurement units N/mm² or MPa. In a stress-strain diagram, the tensile strength is the highest point of the curve. This is mostly determined by a tensile test, as maximum tensile force achieved with respect to an original cross-section of a standardized tensile specimen. The term “predefined” specifically refers to the circumstance that the tensile strength of the locking member is defined prior to using the insertion device. Thus, the tensile strength is predetermined and known prior to using the insertion device. Exemplarily, the tensile strength may be adjusted such as through a choice of material.

As outlined above, the pusher is configured for applying the external force to overcome the predefined tensile strength of the locking member. The term “external force” as used herein is a broad term and is to be given its ordinary and customary meaning to a person of ordinary skill in the art and is not to be limited to a special or customized meaning. The term specifically may refer, without limitation, to an arbitrary force originating from a source outside the insertion device. Exemplarily, the external force may be applied by action of the subject. However, the external force may also be applied by action of a person which differs from the subject. The external force may be applied in a direction of insertion of the insertion component and the medical device. The direction of insertion may correspond to a longitudinal axis of the insertion device. The external force may be applied by the subject or by the person which differs from the subject before insertion of the insertion component and the medical device into the body tissue of the subject. Exemplarily, the external force may be applied to the pusher by pushing or pressing the pusher in a direction of insertion. The term “overcoming the predefined tensile strength of the locking member” may exemplarily refer to applying a force to the locking member such that the locking member breaks or ruptures. Thus, a connection between the medical device holding structure and a further component of the insertion device which may exemplarily a stationary component of the insertion device, exemplarily the insertion device housing, may be disconnected and the medical device holding structure may move relative to stationary components of the insertion device, exemplarily relative to the insertion device housing, exemplarily in the direction of insertion.

As outlined above, the locking member may exemplarily be or may comprise the at least one fiber. The term “fiber” as used herein is a broad term and is to be given its ordinary and customary meaning to a person of ordinary skill in the art and is not to be limited to a special or customized meaning. The term specifically may refer, without limitation, to a linear, elementary structure. The fiber may be a thin, flexible structure in relation to its length. Exemplarily, the fiber may have a ratio of length to diameter of at least 3:1, preferably of at least 10:1. The fiber may be solid or hollow. Exemplarily, the fiber may have a hollow structure. Exemplarily, the locking member may comprise one single fiber. The fiber may exemplarily be a filament. The filament may be a fiber having an arbitrary length. The filament may also be referred to as continuous fiber. The fiber may exemplarily be a monofilament. However, also other embodiments may be feasible. Exemplarily, the locking member may also comprise a bundle of fibers.

Other embodiments of locking members are possible. For example, the locking member may have a sheet form. For example, the locking member may be and/or comprise at least one strip, e.g. made from a foil. A foil can be manufactured with well controlled properties. The foil may be cut in strips of desired width, e.g. with a pre-defined thickness. Width and thickness may define the cross-sectional area and thus the release force.

The locking member may comprise at least one material, which can be injection-molded and/or extruded as filament. The material may be selected depending on the desired release characteristics. Rather brittle materials may be used for a well-defined release. The locking member may comprise at least one plastic material, e.g. at least one polymer. The plastic material may be re-inforced plastics like carbon reinforced or glass fiber reinforced. The locking member may exemplarily comprise at least one material selected from the group consisting of: polycarbonate (PC); polylactic acid (PLA); Ethylene Vinyl Acetate (EVA); Polyetheretherketon (PEEK), but is not limited to this examples.

The predefined release force may be defined by the tensile strength Fₜᵤ of the locking member. The desired release force may define a desired combination of the used (fiber) material, size and number of filaments. For example, for a release force of 25 N and a material with e.g. 50 N/mm², a filament cross section area may amount to 0.5 mm². The predefined tensile strength Fₜᵤ of the locking member may be from 5 N/mm² to 200 N/mm², preferably from 30 N/mm² to 60 N/mm². The cross sectional area may be adjusted to the Fₜᵤ and the desired release force.

Further, the release force of the locking member may be from 20 N to 30 N, for example, 2±1 N.

The locking member, e.g. the fiber, may have a length of a few millimeters.

The fiber may exemplarily be a chemical fiber. The fiber may be manufactured by extrusion. Exemplarily, the fiber may be manufactured by a melt spinning process. The melt spinning process may comprise two steps: a spinning process and a fiber drawing process. At least one material to be processed may be first melted, exemplarily plasticized, optionally mixed with additives and pressed through a spinneret in an extruder. After leaving the spinneret, the fibers may be stretched, i.e. drawn to length, resulting in a tapering of the fibers.

Alternatively, the locking member, exemplarily the fiber may be manufactured by injection molding. Exemplarily, for injection molding a rather complex shape may be used. Optionally, the locking member may be part of the medical device holding structure or of the insertion device housing. Thus, the locking member and the medical device holding structure may be manufactured as one single piece. Alternatively, the locking member and the insertion device housing may be manufactured as one single piece.

Optionally, the locking member may have at least one predetermined breaking point. Thereby, exemplarily, the locking member may be manufactured by injection molding. As further used herein, the term “predetermined breaking point” may refer to an arbitrary part of an element being configured to break during mechanical load while other parts of the element remain undamaged. Exemplarily, the predetermined breaking point may comprise at least one notch wherein a thickness of the element may be smaller in comparison to other parts of the element. Exemplarily, the predetermined breaking point may be a ring-shaped breaking point.

The locking member may comprise one single structure such as one single fiber or filament. Alternatively, the locking member may comprise a plurality of separate structures such as a plurality of separate fibers or filaments. Thus, the locking member may comprise two, three, four or even more separate structures. The separate structures may be arranged in a distance to each other. Thus, exemplarily, the separate structures may not touch each other. For example, the locking member may comprise a plurality of filaments. The use of several filaments can allow creating an axially symmetrical structure. The insertion device may comprise a plurality of mechanical units. If there are some other mechanical units, such as needle retraction mechanism, which are placed in the middle, there may be no space for a filament in the middle. In this case the insertion device may comprise, e.g. equal, filaments symmetrically distributed around the mechanism in the middle. The size of the filaments in case of using a plurality of filaments may be smaller compared to the single fiber. This may allow keeping the release force value. The distance between the filaments can be quantified and may be defined by the overall system design. The maximum distance may not exceed the given insertion device diameter (e.g. if round). The minimum distance may be defined by the mechanism placed in the middle and making it necessary to use several distributed filaments.

In case the locking member comprises a plurality of separate structures, each of these separate structures may have the same length.

The structures, exemplarily the separate structures, of the locking member may be positioned symmetrically with respect a longitudinal axis of the insertion device. Exemplarily, each of the structures may be arranged at a same distance from the longitudinal axis of the insertion device and each of the structures may be arranged uniformly on a virtual radius around the longitudinal axis of the insertion device. Also other arrangements may be feasible. Preferably, the insertion device comprises one filament in the middle. However, as described above other embodiments using a plurality of filaments may be possible. The arranging of the filaments may be symmetric. Thus, by using of three filaments, they may be arranged triangularly symmetrical.

The locking member may have a cross-sectional area depending on the desired release force. Values of the cross-sectional area may result from Fₜᵤ of the used filament material and a desired release force. For example, the locking member, exemplarily the fiber, may have a cross-sectional area from 0.2 mm² to 0.9 mm², preferably from 0.3 mm² to 0.7 mm², more preferably from 0.4 mm² to 0.6 mm². In case the locking member comprises a plurality of separate structures, the structures may have a sum cross-sectional area from 0.2 mm² to 0.9 mm², preferably from 0.3 mm² to 0.7 mm², more preferably from 0.4 mm² to 0.6 mm².

Further, the locking member, exemplarily the fiber, may have a diameter of 0.3 mm to 0.9 mm, preferably of 0.4 mm to 0.7 mm. Exemplarily, the locking member, exemplarily the fiber, may have a diameter of 0.8 mm.

The locking member may be attached to components of the insertion device, exemplarily to the medical device holding structure and/or the insertion device housing by one or more of heat stacking, knotting, adhering, and bonding. Also other methods may be feasible. Exemplarily, the locking member may be attached to the medical device holding structure by melting in free space or by melting in a melting shape.

The medical device holding structure may be guidable within the insertion device, exemplarily within at least component of the insertion device, exemplarily within at least one stationary component of the insertion device. Exemplarily, the medical device holding structure may be guidable within the insertion device housing. Exemplarily, the insertion component and the medical device may be movable with the medical device holding structure. Exemplarily, the medical device and/or the insertion component may be attached to the medical device holding structure. Further, the medical device holding structure may be attached to the pusher.

The insertion device housing may at least partially surround the medical device holding structure. Exemplarily, the insertion device housing may fully surround the receptacle of the medical device holding structure. Further, the insertion device housing may fully surround the insertion component and the medical device. The pusher may at least partially surround the insertion device housing. The insertion device housing may comprise at least one guiding surface, exemplarily a circumferential guiding surface, for guiding the pusher during insertion of the medical device into the body tissue of the subject. Thus, the guiding surface may be an outer surface of the insertion device housing, such as an outer surface having one of a circular cross-section. The pusher may overlap with the insertion device housing within the guiding surface. During insertion, the pusher, such as an inner surface of the pusher, may slide over the guiding surface. The medical device holding structure may have mounting elements. The mounting elements may be configured for connecting or attaching the receptacle of the medical device holding structure to the pusher. Exemplarily, the mounting element may be configured for attaching the receptacle to an inner wall of the pusher. The insertion device housing may have the at least one opening for leading the at least one mounting element from a space enclosed by the insertion device housing to a space enclosed by the pusher.

For inserting the medical device, the pusher, the medical device holding structure and the insertion component and optionally also the medical device may be movable relative to the insertion device housing. As outlined above, the insertion device housing may be a stationary component. Exemplarily, the movement of the pusher, the medical device holding structure and the insertion component optionally also of the medical device relative to the insertion device housing may be a translational movement, exemplarily along the longitudinal axis of the insertion device.

The locking member may comprise at least one first end and at least one opposing second end. The terms “first end” and “second end” may be considered as description without specifying an order and without excluding a possibility that several kinds of first ends and second ends may be present. One of the first end and the second end may be attached to the medical device holding structure. Another one of the first end and the second end may be attached to another component of the insertion device, exemplarily to a stationary component of the insertion device. Exemplarily, the stationary component of the insertion device may be the insertion device housing. Exemplarily, the first end may be attached to at least one insertion device housing surface of the insertion device housing and the second end may be attached to at least one medical device holding structure surface of the medical device holding structure or vice versa.

The insertion device housing surface of the insertion device housing and the medical device holding structure surface of the medical device holding structure may respectively extend transverse, exemplarily essentially perpendicular, to the longitudinal axis of the insertion device. The term “essentially perpendicular” may comprise slight deviations from a perpendicular arrangement such as arrangements which deviate from a perpendicular arrangement by no more than 10 degrees, preferably by no more than 5 degrees.

Exemplarily, the insertion device housing surface of the insertion device housing and the medical device holding structure surface of the medical device holding structure may extend essentially parallel to each other. Thereby, the term “essentially parallel” may refer to a property of the medical device holding structure surface of being parallel to the insertion device housing surface. Exemplarily, the medical device holding structure surface may be exactly parallel to the insertion device housing surface. However, small deviations may be feasible. Exemplarily, the medical device holding structure surface may be arranged at an angle of +/−20°, preferably of +/−10°, more preferably of +/−5° to the insertion device housing surface.

The insertion device housing surface of the insertion device housing and the medical device holding structure surface of the medical device holding structure may face each other.

Exemplarily, insertion device housing surface of the insertion device housing and the medical device holding structure surface of the medical device holding structure may be opposing surfaces. The insertion device housing surface of the insertion device housing and the medical device holding structure surface of the medical device holding structure may be arranged in a distance to each other. The distance may correspond to a length of the locking member.

The medical device holding structure surface may be arranged between the insertion device housing surface and the proximal end of the insertion device housing. The insertion device housing may comprise a distal end opposing the proximal end. The insertion device housing surface may be located at the distal end of the insertion device housing. Exemplarily, the insertion device housing surface may correspond to an inner surface of the insertion device housing facing an interior space enclosed by the insertion device housing. The medical device holding structure surface may be a surface of the receptacle of the medical device holding structure, exemplarily an outer surface of the receptacle of the medical device holding structure facing an exterior space of the receptacle and the interior space enclosed by the insertion device housing. In the direction of insertion, the medical device holding structure surface may be arranged after the insertion device housing surface. The insertion device housing surface may be arranged further away from the proximal end of the insertion device housing than the medical device holding structure surface.

At least one of the insertion device housing surface of the insertion device housing and the medical device holding structure surface of the medical device holding structure may comprise at least one opening, exemplarily at least one cut-out and the locking member may be at least partially received in the opening. Exemplarily, the locking member may be fixedly received in the opening.

In a further aspect of the present invention, an insertion system is disclosed. The insertion system comprises at least one medical device. For possible definitions and options, reference may be made to the disclosure of the medical device according to the present invention. Further, the insertion system comprises at least one insertion device as described above or as will further be described below in more detail for at least partially inserting the medical device into a body tissue of a subject. The medical device is held by the medical device holding structure. Thus, for possible definitions and options, reference may be made to the disclosure of the insertion device according to the present invention.

The term “system” as used herein is a broad term and is to be given its ordinary and customary meaning to a person of ordinary skill in the art and is not to be limited to a special or customized meaning. The term specifically may refer, without limitation, to a group of at least two elements which may interact with each other in order to fulfill at least one common function. The at least two components may be handled independently or may be coupled, connectable or integrable in order to form a common device. The term “insertion system” as used herein is a broad term and is to be given its ordinary and customary meaning to a person of ordinary skill in the art and is not to be limited to a special or customized meaning. The term specifically may refer, without limitation, to a group of at least two elements or components which are capable of interacting with each other in order to perform at least one transcutaneous or subcutaneous insertion of at least one of the two elements or components into a body tissue of a subject, such as by performing an incision or a puncture in a skin of the subject and by transferring the at least one of the two elements or components fully or partially into the body tissue.

In a further aspect of the present invention, a method for at least partially inserting a medical device into the body tissue of a subject is disclosed.

The method comprises using the insertion system as described above or as will further be described below in more detail. Thus, for possible definitions and options, reference may be made to the disclosure of the insertion system according to the present invention.

The method comprises the following steps which exemplarily may be performed in the given order. The method may comprise further method steps which are not listed.

The method comprises the following steps:
a) applying the insertion system to a subject's skin; and
b) overcoming the tensile strength of the locking member by applying an external force to the pusher so that the medical device holding structure, the medical device and the insertion component move from the distal position to the proximal position thereby at least partially inserting the medical device into the body tissue of the subject.

The insertion system, exemplarily the insertion device, may comprise the insertion device housing. In step a), the insertion device housing, e.g. the proximal end of the insertion device housing, may be set in contact with the subjects's skin, exemplarily at an insertion site. Thus, the insertion device housing may be applied to the skin site of the subject.

In step b), the external force to the pusher may be applied by action of the subject. By movement of the pusher from the distal position to the proximal position, the medical device holding structure, the insertion component and the medical device may be moved relative to the insertion device housing from the distal position to the proximal position. During step b), the insertion component may be inserted into the body tissue of the subject, thereby inserting the medical device into the body tissue of the subject. The force may be applied by the subject before the insertion of the insertion component into the body tissue.

In step b) also the pusher may move from the distal position to the proximal position.

Further, the method may comprise the following step:
c) removing the insertion device from the subject's skin, thereby separating the insertion device from the medical device.

During step c), the insertion component may be withdrawn from the body tissue of the subject.

The methods and devices according to the present invention provide a large number of advantages over known methods and devices.

The current invention proposes a different way of a release mechanism which exemplarily is not based on a disposition of some parts pressing against each other as commonly used, but on tearing a fiber.

The medical device holding structure and the insertion device housing may be designed at a way to be kept by a fiber. Applying the external force may lead to the fiber tearing and sudden movement of the medical device holding structure with some momentum. The momentum may be defined by the tensile strength of the used fiber and its cross-sectional area.

The suitable material of the fiber may possess a time-stable and well-defined tensile strength with low dependency on operational temperatures. Furthermore, the material may show a very low strain, which means, its elongation under stress may be possibly short before breaking apart. Materials may be polycarbonate (PC) and polylactic acid (PLA). Both materials commonly show strain in the range of a few percent, low temperature dependence of the tensile strength in the normal temperature range. The typical release force lies in the range of 20 N to 30 N, considering tensile strength of PLA or PC of around 50 MPa, the cross-sectional area of 0.4-0.6 mm² is needed, which correspond to a fiber with around 0.8 mm in diameter. Such fibers can be easily manufactured as well-defined fibers by means of extrusion.

The inventive concept may enable a direct and easy setting of the insertion force via the tensile strength of the locking structure, i.e. via the structure's material and shape/strength.

Furthermore, by the choice of material the insertion force may be set in a very time-stable, well-defined and temperature independent manner.

The invention relates to controlling the insertion force, i.e. to setting the initial speed of the insertion component.

The locking member may have a well-defined tensile strength setting the insertion device release force via the rupture/failure of the locking member upon tension. The locking member may restrict the insertion movement of the insertion component. By applying the external force, the locking member may be tensioned until the insertion process is started

Summarizing and without excluding further possible embodiments, the following embodiments may be envisaged:

Embodiment 1: An insertion device for at least partially inserting a medical device into a body tissue of a subject comprising:
at least one medical device holding structure;
an insertion component movable with the medical device holding structure from a distal position to a proximal position;
at least one locking member having a predefined tensile strength Fₜᵤ, wherein the locking member is configured for holding the medical device holding structure and the insertion component in the distal position; and
at least one pusher configured for applying an external force to overcome the predefined tensile strength of the locking member.

Embodiment 2: The insertion device according to the preceding embodiment, wherein the locking member comprises at least one fiber.

Embodiment 3: The insertion device according to the preceding embodiment, wherein the at least one fiber is manufactured by extrusion.

Embodiment 4: The insertion device according to any one of the two preceding embodiments, wherein the fiber has a hollow structure.

Embodiment 5: The insertion device according to any one of the preceding embodiments, wherein the locking member comprises a plurality of separate structures.

Embodiment 6: The insertion device according to the preceding embodiment, wherein the structures of the locking member are positioned symmetrically with respect a longitudinal axis of the insertion device.

Embodiment 7: The insertion device according to any one of the preceding embodiments, wherein the locking member is attached to the medical device holding structure by one or more of heat stacking, knotting, adhering, and bonding.

Embodiment 8: The insertion device according to any one of the preceding embodiments, wherein the locking member is attached to the medical device holding structure by melting in free space or by melting in a melting shape.

Embodiment 9: The insertion device according to any one of the preceding embodiments, wherein the insertion device comprises at least one insertion device housing.

Embodiment 10: The insertion device according to the preceding embodiment, wherein the locking member is attached to the insertion device housing by one or more of heat stacking, knotting, adhering, and bonding.

Embodiment 11: The insertion device according to any one of the two preceding embodiments, wherein the locking member is attached to the insertion device housing by melting in free space or by melting in a melting shape.

Embodiment 12: The insertion device according to any one of the three preceding embodiments, wherein the medical device holding structure is guidable within the insertion device housing.

Embodiment 13: The insertion device according to any one of the four preceding embodiments, wherein for inserting the medical device, the pusher, the medical device holding structure and the insertion component are movable relative to the insertion device housing.

Embodiment 14: The insertion device according to the preceding embodiment, wherein the movement of the pusher, the medical device holding structure and the insertion component relative to the insertion device housing is a translational movement.

Embodiment 15: The insertion device according to any one of the six preceding embodiments, wherein the pusher at least partially surrounds the insertion device housing.

Embodiment 16: The insertion device according to any one of the seven preceding embodiments, wherein the locking member comprises at least one first end and at least one opposing second end, wherein the first end is attached to at least one insertion device housing surface of the insertion device housing and wherein the second end is attached to at least one medical device holding structure surface of the medical device holding structure or vice versa.

Embodiment 17: The insertion device according to the preceding embodiment, wherein the insertion device housing surface of the insertion device housing and the medical device holding structure surface of the medical device holding structure face each other.

Embodiment 18: The insertion device according to any one of the two preceding embodiments, wherein the insertion device housing surface of the insertion device housing and the medical device holding structure surface of the medical device holding structure are arranged in a distance to each other.

Embodiment 19: The insertion device according to any one of the three preceding embodiments, wherein the insertion device housing surface of the insertion device housing and the medical device holding structure surface of the medical device holding structure extend essentially parallel to each other.

Embodiment 20: The insertion device according to any one of the four preceding embodiments, wherein at least one of the insertion device housing surface of the insertion device housing and the medical device holding structure surface of the medical device holding structure comprises at least one opening, exemplarily at least one cut out, wherein the locking member is at least partially received in the opening.

Embodiment 21: The insertion device according to the preceding embodiment, wherein the locking member is fixedly received in the opening.

Embodiment 22: The insertion device according to any one of the preceding embodiments, wherein the medical device holding structure is at least partially received in the pusher.

Embodiment 23: The insertion device according to any one of the preceding embodiments, wherein the locking member comprises one or more of polycarbonate, polylactic acid, Ethylene Vinyl Acetate, Polyetheretherketon and others.

Embodiment 24: The insertion device according to any one of the preceding embodiments, wherein the predefined tensile strength Fₜᵤ of the locking member is from 5 N/mm² to 200 N/mm², preferably from 30 N/mm² to 60 N/mm².

Embodiment 25: The insertion device according to any one of the preceding embodiments, wherein the release force of the locking member is from 20 N to 30 N.

Embodiment 26: The insertion device according to any one of the preceding claims, wherein the locking member comprises a cross-sectional area from 0.4 mm² to 0.6 mm².

Embodiment 27: The insertion device according to any one of the preceding embodiments, wherein the locking member comprises a diameter of 0.8 mm.

Embodiment 28: The insertion device according to any one of the preceding embodiments, wherein the pusher comprises at least one cap.

Embodiment 29: An insertion system, wherein the insertion system comprises at least one medical device and at least one insertion device according to any one of the preceding embodiments for at least partially inserting the medical device into a body tissue of a subject, wherein the medical device is held by the medical device holding structure.

Embodiment 30: The insertion system according to the preceding embodiment, wherein the medical device holding structure comprises at least one receptacle, wherein the medical device is at least partially received in the receptacle.

Embodiment 31: The insertion system according to any one of the two preceding embodiments, wherein the medical device comprises at least one insertable portion.

Embodiment 32: The insertion system according to any one of the three preceding embodiments, wherein the medical device comprises at least one device selected from the group consisting of: an analyte sensor for detecting at least one analyte in a body fluid of the subject; an infusion cannula; a stimulating electrode.

Embodiment 33: The insertion system according to any one of the four preceding embodiments, wherein the medical device comprises at least one housing, wherein one or more electronic components are received in the housing.

Embodiment 34: A method for at least partially inserting a medical device into the body tissue of a subject, wherein the method comprises using the insertion system according to any one of the preceding embodiments referring to an insertion system, wherein the method comprises the following steps:
a) applying the insertion system to a subject's skin,
b) overcoming the tensile strength of the locking member by applying an external force to the pusher so that the medical device holding structure, the medical device and the insertion component move from the distal position to the proximal position thereby at least partially inserting the medical device into the body tissue of the subject.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIGS. 1A and 1B show an exemplary method according to the present invention for at least partially inserting a medical device into a body tissue of a subject;

FIG. 2 shows a detailed view on at least one locking member of at least one insertion device according to the present invention;

FIGS. 3A and 3B illustrate one option for mounting a locking member in components of an insertion device;

FIGS. 4A to 4C illustrate one further option for mounting a locking member in components of an insertion device;

FIGS. 5A to 5C illustrate one further option for mounting a locking member in components of an insertion device; and

FIGS. 6A to 6C illustrate one further option for mounting a locking member in components of an insertion device.

### DETAILED DESCRIPTION OF THE EMBODIMENTS

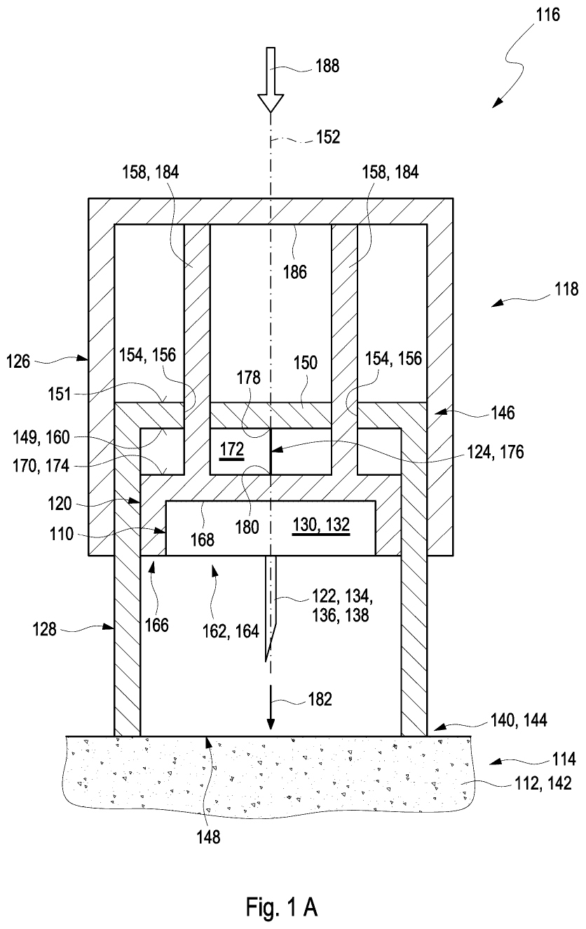

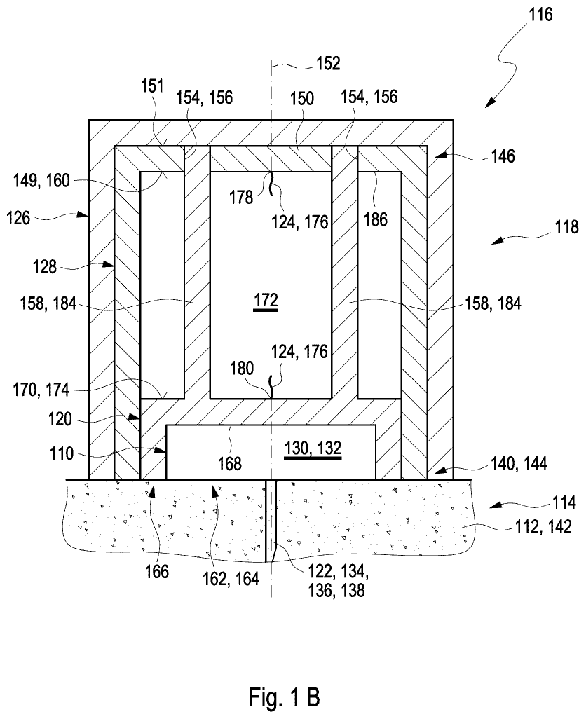

FIGS. 1A and 1B show an exemplary method for at least partially inserting a medical device 110 into a body tissue 112 of a subject 114. FIGS. 1A and 1B respectively show an insertion system 116 having an insertion device 118 and the medical device 110. The insertion system 116 is respectively depicted in a schematic and cross-sectional view.

As illustrated in FIGS. 1A and 1B, the insertion device 118 comprises at least one medical device holding structure 120. The medical device 110 is held by the medical device holding structure 120. Further, the insertion device 118 comprises an insertion component 122 movable with the medical device holding structure 120 from a distal position to a proximal position. Further, the insertion device 118 comprises at least one locking member 124 having a predefined tensile strength Fₜᵤ. The locking member 124 is configured for holding the medical device holding structure 120 and the insertion component 122 in the distal position such as illustrated in FIG. 1A. Further, the insertion device 118 comprises at least one pusher 126 configured for applying an external force to overcome the predefined tensile strength of the locking member 124. Further, the insertion device 118 may comprise at least one insertion device housing 128.

In FIG. 1A, the insertion system 116 is shown wherein the insertion component 122, the medical device holding structure 120, the medical device 110 and the pusher 126 are in the distal position. In FIG. 1B, the insertion system 116 is shown wherein the insertion component 122, the medical device holding structure 120, the medical device 110 and the pusher 126 are in the proximal position.

The medical device 110 may comprise at least one component which may be configured to stay outside of the body tissue 112. Exemplarily, the medical device 110 may comprise at least one housing 130. One or more electronic components may be received in the housing 130. The housing 130 may also be referred to as electronics unit 132. Further, the medical device 110 may comprise at least one insertable portion 134. The medical device 110 may exemplarily comprise at least one analyte sensor 136 for detecting at least one analyte in a body fluid of the subject 114.

The insertion component 122 may be configured for supporting the insertion of the insertable portion 134 of the medical device 110. The insertion component 122 may be or may comprise an insertion cannula 138. The insertable portion 134 of the medical device 110 may be at least partially received in a lumen of the insertion cannula 138.

The pusher 126 may surround and/or may enclose fully or partially one or more further components, such as the medical device holding structure 120, the medical device 110 and the locking member 124. Exemplarily, the medical device holding structure 120, the medical device 110 and/or the locking member 124 may be at least partially received in the pusher 126.

Further, the insertion device housing 128 may be partially enclosed by the pusher 126. The insertion device housing 128 may be essentially rotationally symmetric, exemplarily in accordance with the symmetry of the pusher 126 of the insertion device 118. The insertion device housing 128 may be stationary with respect to the pusher 126 of the insertion device 118. For example, when using the insertion device 118, the pusher 126 may be configured for sliding into the insertion device housing 128. The insertion device housing 128, e.g. a proximal end 140 of the insertion device housing 128, may be in contact with the subject's skin 142 when the insertion device 118 is used.

The insertion device housing 128 may be at least partially formed as a hollow cylinder having a first end 144 and an opposing second end 146. The insertion device housing 128 may be configured for getting in contact with the skin 142 of the subject 114 via the first end 144. At the first end 144, the insertion device housing 128 may have an opening 148. The medical device 110 may get in contact with the skin 142 of the subject 114 via the opening 148. At the second end 146, the insertion device 118 may be at least partially closed. At the second end 146, the insertion device 118 may have a wall 150 which may extend transverse, exemplarily essentially perpendicular, to a longitudinal axis 152 of the insertion device 118. The wall 150 may have one or more openings 154, exemplarily one or more through holes 156 for leading sections 158 of the medical device holding structure 120 from one side 149 of the wall 150 to another opposing side 151 of the wall 150. The wall 150 be may comprise an insertion device housing surface 160 which will further be described below in more detail.

The medical device holding structure 120 may comprise at least one receptacle 162 and the medical device 110 may be at least partially received in the receptacle 162. For example, medical device holding structure 120 may comprise a rotational symmetric hollow center 164. The rotational symmetric hollow center 164 may exemplarily be open to a proximal end 166 of the medical device holding structure 120. The medical device 110 may be arrangable at the proximal end 166 of the medical device holding structure 120. The receptacle 162 may comprise an inner side 168 facing the medical device 110 and an opposing outer side 170 facing an interior space 172 of the pusher 126 and an interior space 172 of the insertion device housing 128. The outer side 170 may have a medical device holding structure surface 174 which will further be described below in more detail.

The locking member 124 may comprise at least one fiber 176. In the embodiment according to FIGS. 1A and 1B, the locking member 124 may comprise at least one single fiber 176. The locking member 124 may be positioned along the longitudinal axis 152 of the insertion device 118 or of the insertion system 116.

The locking member 124 may be attached to the medical device holding structure 120. Further, the locking member 124 may be attached to the insertion device housing 128.

The locking member 124 may comprise at least one first end 178 and at least one opposing second end 180. The first end 178 may be attached to the at least one insertion device housing surface 160 of the insertion device housing 128 and the second end 180 may be attached to at least one medical device holding structure surface 174 of the medical device holding structure 120 or vice versa.

The insertion device housing surface 160 of the insertion device housing 128 and the medical device holding structure surface 174 of the medical device holding structure 120 may face each other and may be arranged in a distance to each other. Further, the insertion device housing surface 160 of the insertion device housing 128 and the medical device holding structure surface 174 of the medical device holding structure 120 may extend essentially parallel to each other. Further, the insertion device housing surface 160 of the insertion device housing 128 and the medical device holding structure surface 174 of the medical device holding structure 120 may extend essentially perpendicular to the longitudinal axis 152 of the insertion device 118 which may be equivalent to a direction of insertion 182 of the medical device 110.

As outlined above, the medical device holding structure 120 may comprise the at least one receptacle 162 configured for holding the medical device 110. Further, the medical device holding structure 120 may have at least one mounting element 184 for mounting the receptacle into the pusher 126, exemplarily into an interior space of the pusher 126. Exemplarily, the mounting element 184 may be configured for attaching the receptacle 162 to an inner wall 186 of the pusher 126. The insertion device housing 128 may have the at least one opening 154 for leading the at least one mounting element 184 from a space enclosed by the insertion device housing 128 to a space enclosed by the pusher 126.

As illustrated in FIG. 1A, the insertion system 116 is applied to the subject's skin 142. Thereby, the pusher 126, the medical device holding structure 120, the medical device 110 and the insertion component 122 are in the distal position. The locking member 124 is configured to hold the medical device holding structure 120, the insertion component 122 and the medical device 110 in the distal position.

As further illustrated in FIG. 1A with arrow 188, an external force may be applied to the pusher 126 such that a tensile strength of the locking member 124 is overcome. Thus, as illustrated in FIG. 1B, the medical device holding structure 120, the medical device 110 and the insertion component 122 move from the distal position to the proximal position thereby at least partially inserting the medical device 110 into the body tissue 112 of the subject 114.

FIGS. 1A and 1B represent an oversimplified, schematically depiction of the insertion system 116 and the proposal insertion mechanism. An insertion cannula retraction mechanism is not illustrated. An acting principle is represented.

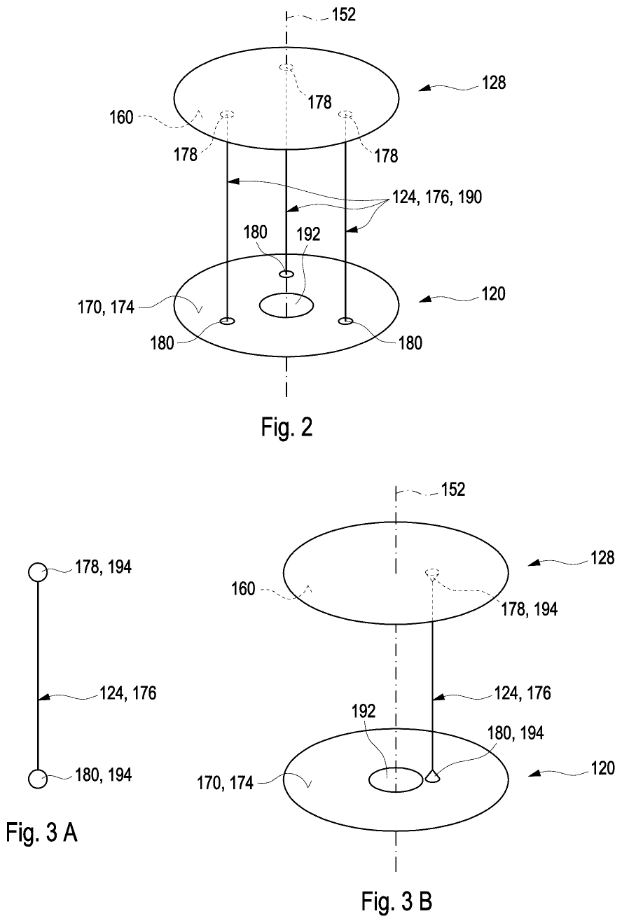

FIG. 2 shows a detailed view on at least one locking member 124 of at least one insertion device 118 according to the present invention. The insertion device 118 may correspond at least partially to the insertion device 118 as illustrated in FIGS. 1A and 1B. Thus, reference to the description of FIGS. 1A and 1B above may be made.

In FIG. 2, the insertion device housing surface 160 of the insertion device housing 128 and the medical device holding structure surface 174 of the medical device holding structure 120 are illustrated. The locking member 124 may comprise the fiber 176. The locking member 124 may comprise the first end 178 and the opposing second end 180. The first end 178 may be attached to the insertion device housing surface 160 of the insertion device housing 128 and the second end 180 may be attached to the medical device holding structure surface 174 of the medical device holding structure 120 or vice versa.

The number of locking members 124 may be more than one, and their placement may be varied. One important parameter may be a sum cross-sectional area of the locking members 124.

As illustrated in FIG. 2, the locking member 124 may comprise a plurality of separate structures 190, exemplarily of separate fibers 176. In the embodiment according to FIG. 2, the locking member 124 may comprise three separate fibers 176. The structures 190 may be positioned symmetrically with respect to the longitudinal axis 152 of the insertion device 118. The fibers 176 may also have a hollow structure and/or a non-round shape.

In FIG. 2 an opening 192 of the medical device holding structure 120 is illustrated which refers to a technical opening or mechanism.

There are many different ways, how to mount the locking member 124 in the mechanical components. Several examples are described in the following.

FIGS. 3A and 3B illustrate one option for mounting a locking member 124 in components of an insertion device 118. The insertion device 118 may correspond at least partially to the insertion device 118 as illustrated in FIGS. 1A and 1B. Thus, reference to the description of FIGS. 1A and 1B above may be made.

As illustrated in FIG. 3A, the locking member 124 may comprise a fiber 176 and the locking member 124 may comprise the first end 178 and the opposing second end 180. The locking member 124 may be melted locally at the first end 178 and at least second end 180. The melting may be performed either in free space such as illustrated in FIG. 3A. Thus, spheres 194 may be formed. Further, the melting may be performed in a melting shape. Thus, any desired shape may be created.

In FIG. 3B, the insertion device housing surface 160 of the insertion device housing 128 and the medical device holding structure surface 174 of the medical device holding structure 120 are illustrated. The first end 178 may be attached to the insertion device housing surface 160 of the insertion device housing 128 and the second end 180 may be attached to the medical device holding structure surface 174 of the medical device holding structure 120 or vice versa.

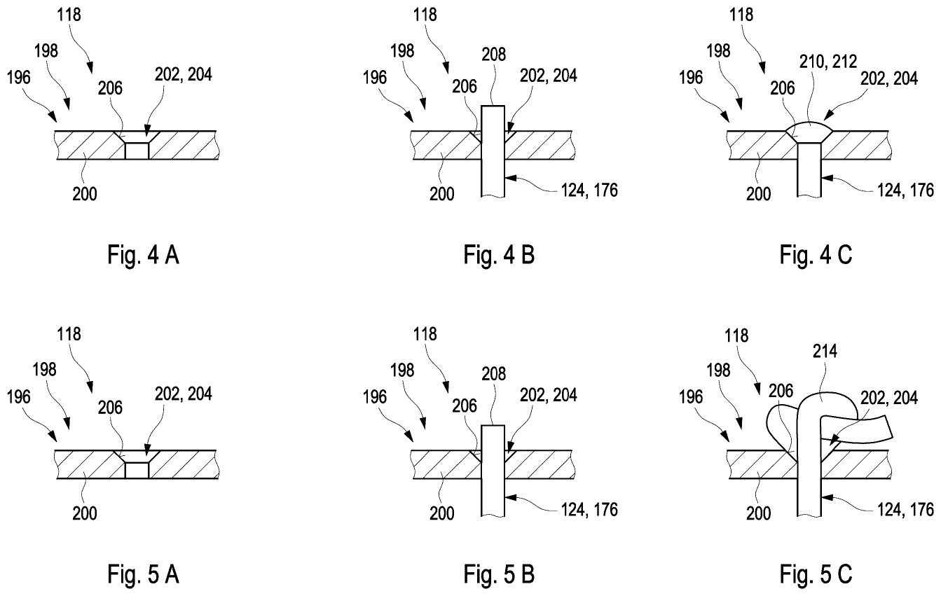

FIGS. 4A to 4C illustrate one further option for mounting a locking member 124 in components of an insertion device 118. The insertion device 118 may correspond at least partially to the insertion device 118 as illustrated in FIGS. 1A and 1B. Thus, reference to the description of FIGS. 1A and 1B above may be made.

In FIGS. 4A to 4C one section 196 of a component 198 of the insertion device 118 where the locking member 124 is attached to, exemplarily a wall 200 of the section 196 of the component 198, is shown. The component 198 may exemplarily be the medical device holding structure 120, exemplarily a wall of the receptacle 162 of the medical device holding structure 120, or of the insertion device housing 128.

The wall 200 of the section 196 of the component 198 may comprise at least one opening 202, exemplarily at least one cut-out 204. The cut-out 204 may have at least one beveled surface 206. The cut-out 204 may allow mounting and fixation of an end 208 of the locking member 124. The locking member 124 may be fixed within the cut-out 204 using an adhesive.

A forming of a locking member tip 210, exemplarily of a fiber tip 212, as illustrated in FIGS. 4B and 4C may be performed directly while mounting the locking member 124 in the mechanical components using e.g. heat stacking. Alternatively, the forming of the locking member tip 210 may be performed prior to mounting.

FIGS. 5A to 5C illustrate one further option for mounting a locking member 124 in components of an insertion device 118. The insertion device 118 may correspond at least partially to the insertion device 118 as illustrated in FIGS. 1A and 1B. Thus, reference to the description of FIGS. 1A and 1B above may be made.

In FIGS. 5A to 5C the section 196 of the component 198 of the insertion device 118 where the locking member 124 is attached to, exemplarily the wall 200 of the section 196 of the component 198, is shown. The component 198 may exemplarily be the medical device holding structure 120, exemplarily a wall of the receptacle 162 of the medical device holding structure 120, or of the insertion device housing 128.

The wall 200 of the section of the component 198 may comprise the at least one opening 202, exemplarily the at least one cut-out 204. The cut-out may 204 have the beveled surface 206. The cut-out 204 may allow mounting and fixation of the end 208 of the locking member 124.

As illustrated in FIG. 5B, the locking member 124, exemplarily the fiber 176, may be guided through the opening 202 and, as illustrated in FIG. 5C, on one side of the wall 200 a knot 214 may be formed with the locking member 124.

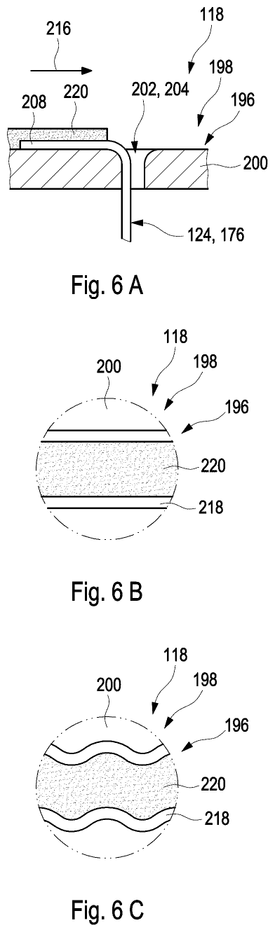

FIGS. 6A to 6C illustrate one further option for mounting a locking member 124 in components of an insertion device 118. The insertion device 118 may correspond at least partially to the insertion device 118 as illustrated in FIGS. 1A and 1B. Thus, reference to the description of FIGS. 1A and 1B above may be made.

In FIGS. 6A to 6C, the section 196 of the component 198 of the insertion device 118 where the locking member 124 is attached to, exemplarily the wall 200 of the section 196 of the component 198, is shown. The component 198 may exemplarily be the medical device holding structure 120, exemplarily a wall of the receptacle 162 of the medical device holding structure 120, or of the insertion device housing 128. FIG. 6A shows a cross-sectional view of the wall 200 whereas 6B and 6C respectively show the wall 200 in a top view.

The wall 200 may comprise the at least one opening 202, exemplarily the at least one cut-out 204. As illustrated in FIG. 6A, the locking member 124, exemplarily the fiber 176, may be guided through the opening 202. The fiber 176 may be bent, exemplarily in an angle of 90° to a direction of extension 216 of the wall 200. The fiber 176 may be fixed by means of adhesive 220.

As can be seen in the top views of the wall 200 in FIGS. 6B and 6C, the wall 200 may have at least one groove 218 which may be configured for receiving the fiber 176, exemplarily the end 208 of the fiber 176. The adhesive 220 may be applied within the groove 218. The groove 218 may be configured for enhancing a contact surface area between the wall 200 and the fiber 176.

In the embodiment according to FIG. 6B, the groove 218 may have a straight shape.

In the embodiment according to FIG. 6C, the groove 218 may have a curved shape. Due to the curved shape, an enhancing of the contact surface area between the wall 200 and the fiber 176 may be even more increased. Further, due to the curved shape a fixation of the fiber 176 within the groove 218 may be improved.

List of reference numbers

| | |
|---|---|
| 110 | medical device |
| 112 | body tissue |
| 114 | subject |
| 116 | insertion system |
| 118 | insertion device |
| 120 | medical device holding structure |
| 122 | insertion component |
| 124 | locking member |
| 126 | pusher |
| 128 | insertion device housing |
| 130 | housing |
| 132 | electronics unit |
| 134 | insertable portion |
| 136 | analyte sensor |
| 138 | insertion cannula |
| 140 | proximal end |
| 142 | skin |
| 144 | first end |
| 146 | second end |
| 148 | opening |
| 149 | side |
| 150 | wall |
| 151 | side |
| 152 | longitudinal axis |
| 154 | opening |
| 156 | through hole |
| 158 | section |
| 160 | insertion device housing surface |
| 162 | receptacle |
| 164 | rotational symmetric hollow center |
| 166 | proximal end |
| 168 | inner side |
| 170 | outer side |
| 172 | interior space |
| 174 | medical device holding structure surface |
| 176 | fiber |
| 178 | first end |
| 180 | second end |
| 182 | direction of insertion |
| 184 | mounting element |
| 186 | inner wall |
| 188 | arrow |
| 190 | structure |
| 192 | opening |
| 194 | sphere |
| 196 | section |
| 198 | component |
| 200 | wall |
| 202 | opening |
| 204 | cut-out |
| 206 | beveled surface |
| 208 | end |
| 210 | locking member tip |
| 212 | fiber tip |
| 214 | knot |
| 216 | direction of extenstion |
| 218 | groove |
| 220 | adhesive |

1. An insertion device comprising:
at least one medical device holding structure;
an insertion component movable with the medical device holding structure from a distal position to a proximal position;
at least one locking member having a predefined tensile strength Fₜᵤ, wherein the locking member is configured for holding the medical device holding structure and the insertion component in the distal position; and
at least one pusher configured for applying an external force to overcome the predefined tensile strength of the locking member.

2. The insertion device of claim 1, wherein the at least one locking member comprises at least one fiber.

3. The insertion device of claim 1, wherein the at least one locking member comprises a plurality of separate structures.

4. The insertion device of claim 3, wherein the plurality of separate structures of the at least one locking member are positioned symmetrically with respect a longitudinal axis of the insertion device.

5. The insertion device of claim 1, wherein the at least one locking member is attached to the at least one medical device holding structure by one or more of heat stacking, knotting, adhering, and bonding.

6. The insertion device of claim 1, further comprising at least one insertion device housing and wherein the medical device holding structure is guidable within the insertion device housing, and wherein the at least one locking member is attached to the insertion device housing by one or more of heat stacking, knotting, adhering, and bonding.

7. The insertion device of claim 1, wherein:
the insertion device is used for at least partially inserting a medical device into a body tissue of a subject,
the medical device, the pusher, the medical device holding structure and the insertion component are movable relative to the insertion device housing, and
the movement of the pusher, the medical device holding structure and the insertion component relative to the insertion device housing is a translational movement.

8. The insertion device of claim 6, wherein:
the at least one locking member comprises at least one first end and at least one opposing second end,
the first end is attached to at least one insertion device housing surface of the insertion device housing and the second end is attached to at least one medical device holding structure surface of the medical device holding structure, or
the first end is attached to at least one medical device holding structure surface of the medical device holding structure and the second end is attached to at least one insertion device housing surface of the insertion device housing.

9. The insertion device of claim 1, wherein the at least one locking member comprises at least one material selected from the group consisting of: polycarbonate, polylactic acid, Ethylene Vinyl Acetate, or Polyetheretherketon.

10. The insertion device of claim 1, wherein the predefined tensile strength Fₜᵤ of the at least one locking member is from 5 N/mm² to 200 N/mm².

11. The insertion device of claim 1, wherein the at least one locking member has a release force from 20 N to 30 N.

12. The insertion device of claim 1, wherein the at least one locking member comprises a cross-sectional area from 0.4 mm² to 0.6 mm².

13. The insertion device of claim 1, wherein the at least one locking member comprises a diameter from 0.3 mm to 0.9 mm.

14. An insertion system comprising: at least one medical device and at least one insertion device according to claim 1 for at least partially inserting the at least one medical device into a body tissue of a subject, wherein the at least one medical device is held by the medical device holding structure.

15. A method for at least partially inserting a medical device into a body tissue of a subject, wherein the method comprises using the insertion system of claim 14, wherein the method comprises the following steps:
a) applying the insertion system to a subject's skin, and
b) overcoming a tensile strength of at least one locking member by applying an external force to a pusher so that the medical device holding structure, the at least one medical device and the insertion component move from the distal position to the proximal position thereby at least partially inserting the at least one medical device into the body tissue of the subject.
